SEQUENCE LISTING (10 sequences)

SEQ ID NO: 1 - DNA, 1275 bp, Homo sapiens
gtctccgcctgcaggtgcagacatctggaggagagagtcggagagcagaaaccacttggctcccagacaattcccctacaggctttgggcctggaattgaggagaaagtgagctaagttggggtggggtgagtccaaagaagcacgggctgggccaagctaagctgctctgggctgggctgatccctccccactcaggggcgggaccccaggaggagggagaggacagagccactgcagaggaccagactgggaaaacaacgatatggcaggagccagtcttggggcccgcttctaccggcagatcaaaagacatccggggatcatcccgatgatcggcttaatctgcctgggcatgggcagcgctgcgctttacttgctgcgactcgcccttcgcagccccgacgtctgctgggacagaaagaacaacccggagccctggaaccgcctgagccccaatgaccaatacaagttccttgcagtttccactgactataagaagctgaagaaggaccggccagacttctaagccaggctgggctgccagtgccatgcaagccacagccagccagcccatccacttcttccactcctccccgcaggccccaaggcatcactccggccaccctgtcccgctactgcttacacaggccgggttcccacgcagaggggaggctgctccacccctactctcctcccttgctcccagcagcggaagcgcctctgacccttggcttgagtcccacgtgggggaggaggaggcaggcagcaccagcaggggtccaccaagagcccagaccagcccctctgccctcctacccgggcctcgaagggtgtggcacaggctacgtgttgagcgtggcctacgtgagccaacaagaagcaggggcctctgagtgccaagcgacgtggcgggctccacgttagcccaggctctgagagccagcccaggggcggcgctgctcagcttgggctggtccagggcctgcccaggctggggcacctttgcctcctgaggcgcagcgcactcctcccctgcccaagcctactgcctcccgctgccgccagtaccccctccagccccacacctgggcctccccctgccactcccctcccttgctcccctctgtccccagggatcaaacagaagcagccgtgggcaaaatacaatttcatttaacaaattgaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 87 aa, Homo sapiens
MAGASLGARFYRQIKRHPGIIPMIGLICLGMGSAALYLLRLALRSPDVCWDRKNNPEPWNRLSPNDQYKFLAVSTDYKKLKKDRPDF

SEQ ID NO: 3 - DNA, 1967 bp, Homo sapiens
ataaaaaccgtcctcgggcgcggcggggagaagccgagctgagcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggctgcgcgaacacgcggagcgcacccgcagtcagctgagcgcgctggagcggcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccgttccctgcctgggcaggggctccaaggaggggccatctggaaacttgtggacagagaagaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacacttaaaaaaaaaaaaaaaaaaaaaaaaaaaaaaaaaaaaaaaaaaaaaaaaaaaaaaaaaaaaaaaa

SEQ ID NO: 4 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 5 - DNA, 2722 bp, Homo sapiens
gagtctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcaaggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttattcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctacattagcattctaa

SEQ ID NO: 6 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 7 - DNA, 1331 bp, Homo sapiens
cctgcatctttttggaaggattctttttataaatcagaaagtgttcgaggttcaaaggtttgcctcggagcgtgtgaacattcctccgctcggttttcaactcgcctccaacctgcgccgcccggccagcatgtctccccgcccgtgaagcggggctgccgcctccctgccgctccggctgccactaacgacccgccctcgccgccacctggccctcctgatcgacgacacacgcacttgaaacttgttctcagggtgtgtggaatcaactttccggaagcaaccagcccaccagaggaggtcccgagcgcgagcggagacgatgcagcggagactggttcagcagtggagcgtcgcggtgttcctgctgagctacgcggtgccctcctgcgggcgctcggtggagggtctcagccgccgcctcaaaagagctgtgtctgaacatcagctcctccatgacaaggggaagtccatccaagatttacggcgacgattcttccttcaccatctgatcgcagaaatccacacagctgaaatcagagctacctcggaggtgtcccctaactccaagccctctcccaacacaaagaaccaccccgtccgatttgggtctgatgatgagggcagatacctaactcaggaaactaacaaggtggagacgtacaaagagcagccgctcaagacacctgggaagaaaaagaaaggcaagcccgggaaacgcaaggagcaggaaaagaaaaaacggcgaactcgctctgcctggttagactctggagtgactgggagtgggctagaaggggaccacctgtctgacacctccacaacgtcgctggagctcgattcacggaggcattgaaattttcagcagagaccttccaaggacatattgcaggattctgtaatagtgaacatatggaaagtattagaaatatttattgtctgtaaatactgtaaatgcattggaataaaactgtctcccccattgctctatgaaactgcacattggtcattgtgaatattttttttttgccaaggctaatccaattattattatcacatttaccataatttattttgtccattgatgtatttattttgtaaatgtatcttggtgctgctgaatttctatattttttgtaacataatgcactttagatatacatatcaagtatgttgataaatgacacaatgaagtgtctctattttgtggttgattttaatgaatgcctaaatataattatccaaattgattttcctttgtgcatgtaaaaataacagtattttaaatttgtaaagaatgtctaataaaatataatctaattacatcatga

SEQ ID NO: 8 - protein, 177 aa, Homo sapiens
MQRRLVQQWSVAVFLLSYAVPSCGRSVEGLSRRLKRAVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

SEQ ID NO: 9 - DNA, 1956 bp, Homo sapiens
ctctctccagctccagctggacctgaagtctcagagctgccaccagcagcaggctcagacactgggctcccagctggggactgctccatggccatggagatagacagcaggcctggggggctccccggcagtagctgcaacctaggtgcagcccgagaacacatgcaggcggtcacccgaaactacatcacccacccccgtgtcacctacaggactgtgtgcagcgtgaacgggcccctggtggtgctggaccgggtcaagtttgcccagtatgcggagatcgtccacttcaccctcccagatgggactcagaggagcgggcaggtgcttgaggtggctggcaccaaggcgattgttcaggtgtttgaagggacatcagggatcgatgccaggaagaccacttgcgaatttacaggggacatcctacgaactccggtgtcagaggacatgctgggtcgggttttcaatggctccggcaagcccattgacaaggggccagtggtcatggcggaggactttctggatatcaatggccagcccatcaacccgcactcccgcatctaccccgaggagatgattcagacgggcatttctcctattgacgtcatgaacagcattgcccgcggccagaagatccccatcttctcagcagccgggctcccccacaatgagattgccgctcagatctgccgccaggcggggctggtgaagaagtccaaggctgtgctggattaccatgacgacaacttcgccatcgtctttgcagccatgggggtgaacatggagacagccagattcttcaagtctgactttgagcagaatggaaccatggggaacgtctgcctcttcctgaacttggccaatgaccccacgatcgagcggatcatcaccccgcgcctggcgctgaccactgctgaattccttgcctaccagtgtgagaagcatgtgctggtcatactgacggacatgagttcctatgcagaggccttgcgggaggtctctgctgctagagaggaggtgcctgggcgccgagggtttcctggatatatgtacacagacctggccaccatctacgagcgggcgggccgcgtggagggtcggggaggatccatcacacagatccccatcctcaccatgcccaacgacgatatcacccaccctatcccagacttgacgggcttcatcacagagggacagatctacgtggacagacagcttcacaacagacagatctacccccccatcaacgtgctcccttccctgtcgcggctgatgaagtcagccattggggaaggcatgacaagaaaggaccatggagatgtctccaaccagctgtacgcctgctatgccatcgggaaggacgtgcaggccatgaaggcagtagttggggaggaggcgctcacctctgaggacctgctctacctggaattcctgcagaagtttgagaagaacttcatcaatcagggcccctacgagaaccgctcggtgttcgagtcgctggacctgggctggaagctgctgcgcatcttccccaaggagatgctgaagcgcattccgcaggccgtgatcgacgagttctattcccgcgagggggcgctgcaggacctcgcgcctgacactgcgctctagccccgcgcgccgtggcaccccaacaccggcagggaacctaccctcggctcccgggtctcccctccctcgccaccccaaccagcggcttctgcgccgccctccgccctccgctggctccgaggtggtgggggcgccgcacgctccatccctttccctcgctcgattccttttcccgcgctccatgcctccccctcgactcccggtgctgcggaagaactgaaggttgcgatgccttactctgacgggagcatctgtatttttatgttaaaagcccacaaaataaaaataaaaagtaactgagatgaatttaaaaaaaaaaaaaaaaaa

SEQ ID NO: 10 - protein, 513 aa, Homo sapiens
MAMEIDSRPGGLPGSSCNLGAAREHMQAVTRNYITHPRVTYRTVCSVNGPLVVLDRVKFAQYAEIVHFTLPDGTQRSGQVLEVAGTKAIVQVFEGTSGIDARKTTCEFTGDILRTPVSEDMLGRVFNGSGKPIDKGPVVMAEDFLDINGQPINPHSRIYPEEMIQTGISPIDVMNSIARGQKIPIFSAAGLPHNEIAAQICRQAGLVKKSKAVLDYHDDNFAIVFAAMGVNMETARFFKSDFEQNGTMGNVCLFLNLANDPTIERIITPRLALTTAEFLAYQCEKHVLVILTDMSSYAEALREVSAAREEVPGRRGFPGYMYTDLATIYERAGRVEGRGGSITQIPILTMPNDDITHPIPDLTGFITEGQIYVDRQLHNRQIYPPINVLPSLSRLMKSAIGEGMTRKDHGDVSNQLYACYAIGKDVQAMKAVVGEEALTSEDLLYLEFLQKFEKNFINQGPYENRSVFESLDLGWKLLRIFPKEMLKRIPQAVIDEFYSREGALQDLAPDTAL

(19) United States
(12) Patent Application Publication
(10) Pub. No.: US 2012/0142553 A1
(43) Pub. Date: Jun. 7, 2012

(54) Molecular Markers in Kidney Cancer

(76) Inventors: Franciscus Petrus Smit, Nijmegen (NL); Jack A. Schalken, Nijmegen (NL)

(21) Appl. No.: 13/379,823

(22) PCT Filed: Jun. 22, 2010
(86) PCT No.: PCT/EP2010/058782

§ 371 Date: Feb. 17, 2012

(30) Foreign Application Priority Data
Jun. 26, 2009 (EP) PCT/EP2009/058059

(51) Int. Cl. C40B 30/04; C12Q 1/68

(52) U.S. Cl. 506/9; 435/6.12

(57) ABSTRACT

The present invention relates to methods for establishing the presence, or absence, of a kidney, or renal, tumour in a human individual suspected of suffering from kidney, or renal, cancer. Specifically, the present invention relates to methods for establishing the presence, or absence, of a kidney tumour in a human individual suspected of suffering from kidney cancer comprising: a) determining the expression of one or more genes chosen from the group consisting of NDUFA4l2, ANGPTL4, EGLN3, PTHLH, and ATP6V1B1 in a sample originating from said human individual; b) establishing up, or down, regulation of expression of said one or more genes as compared to expression of said respective one or more genes in a sample originating from said human individual not comprising kidney tumour cells or tissue, or from an individual, or group of individuals, not suffering from kidney cancer; and c) establishing the presence, or absence, of a kidney tumour based on the established up- or down regulation of said one or more genes.

The present invention relates to methods for establishing the presence, or absence, of a kidney, or renal, tumour in a human individual suspected of suffering from kidney, or renal, cancer. The present invention further relates to the use of the expression of the present genes for establishing the presence, or absence, of a kidney, or renal, tumour in a human individual suspected of suffering from kidney, or renal, cancer and to kit of parts for establishing the presence, or absence, of a kidney, or renal, tumour in a human individual suspected of suffering from kidney, or renal, cancer.

In 2009 there will be 57,760 newly diagnosed cases of renal cell carcinoma (RCC) with 12,980 deaths, according to the American Cancer Society. RCC accounts for 4% of all malignancies. Renal cell carcinoma is the predominant form of kidney, or renal, cancer and the most common forms are clear cell (80%) and papillary (10-15%) renal cell carcinomas. Other forms of kidney, or renal, cancer are transitional cell carcinoma and sarcoma.

The incidence of RCC in male is 67% higher than in female. The incidence of RCC is greatest in developed societies, perhaps due to highly prevalent risk factors such as cigarette smoking, obesity, hypertension, and analgesic use. Lipid peroxidation has been proposed as a unifying etiologic mechanism for these risk factors.

Presently, due to frequent use of abdominal imaging, many cases are discovered incidentally at organ-confined stage.

Open total nephrectomy is a standard therapy for localized RCC but laparoscopic surgery, with nephron-sparing tumor resections or ablations, is used increasingly for small (<4 cm) lesions, and other clinical situations.

Although surgery is potentially curative for organ-confined RCC, one-third of such lesions metastasize after therapy. Furthermore, despite trends toward early diagnosis, 40% of cases develop extra-renal growth or metastases.

RCC is highly variable in terms of clinical behavior. Pathologic stage using the Tumor-Node-Metastasis (TNM) system is a critical prognostic factor and recent refinements to the TNM classification have been proposed to optimize correlation between outcome and tumor size and local extension.

In an attempt to further improve prediction, several groups have developed prognostic models for metastatic or post-nephrectomy RCC. These models combine pathologic findings with clinical parameters such as performance status and laboratory values.

In addition to these clinical models, elevations in immune markers such as erythrocyte sedimentation rate and C-reactive protein have been shown to carry negative prognostic significance. Also perioperative thrombocytosis is a negative prognostic factor in RCC.

Metastatic disease from RCC typically manifests in the lung, bone, brain, abdominal viscera, the contralateral kidney, adrenal glands, and regional lymph nodes. However, RCC may metastasize to unusual locations and present as metastatic carcinoma of unknown primary. Overall survival may correlate with site of metastasis.

Currently, a need exists for biomarkers to predict a biologic propensity for metastasis and likely sites of spread, to provide patients with accurate prognosis, tailor surveillance to detect early relapse in patients at risk, and design targeted molecular therapy.

Renal tumor subtypes are associated with distinct, reoccurring cytogenetic abnormalities and hereditary cancer syndromes. Hereditary tumors often occur multifocally at earlier age, and suspected cases can be diagnosed with a range of clinical genetic assays on patient germline. In contrast, sporadic RCC is tested only infrequently by cytogenetics due to technical difficulty and limited diagnostic sensitivity.

A tumor biomarker can be defined as a surrogate indicator that increases, or decreases, the clinician's suspicion to cancer susceptibility, onset, progression, or recurrence and whether a specific treatment will decrease the risk of such events. There are currently no established tumor markers for RCC in clinical practice; tumor size and stage offer the only viable tools to predict prognosis. A number of molecular markers have been investigated, and although many show clinical potential, none has gained approved clinical application.

For example, lack of B7H1 and B7H4 expression is a strong predictor of overall survival in patients without metastases. Another potentially important marker is IMPS.

While data from clinical trials provide general guidelines for the best 1st and 2nd line therapies for metastatic RCC, these are not always the best choices for each individual patient. There are very few biomarkers that can guide clinicians in the choice of therapy for each individual patient.

In patients with clear cell RCC, responses to IL-2 were associated with the presence of alveolar features in more than 50 percent of the sample, and an absence of papillary features or granular features. Carbonic anhydrase IX (CAIX) expression is HIF dependent and its expression is increased in VHL mutated RCC. High levels of CAIX expression are associated with a more favorable prognosis and a greater likelihood of a response to IL-2.

There are no biomarkers available to predicting responsiveness to molecularly targeted agents. Measurements of VEGF and the soluble VEGF receptor do change in response to treatment but whether such alterations can be used, as a marker for tumor responsiveness remains unknown.

Considering the above, there is a need in the art for improved markers and recent developments in the field of molecular techniques have provided new tools that have led and may lead to the discovery, or identification, of suitable biomarkers. A suitable marker preferably fulfills the following criteria: 1) it must be reproducible (intra- and inter-institutional) and 2) it must have an impact on clinical management.

It is an object of the present invention, amongst other objects, to meet at least partially, if not completely, the above object.

According to the present invention, the above object, amongst other objects, is met by kidney, or renal, tumour markers and methods as outlined in the appended claims.

Specifically, the above object, amongst other objects, is met by a method for establishing the presence, or absence, of a kidney, or renal, tumour in a human individual suspected of suffering from kidney, or renal, cancer comprising:
a) determining the expression of one or more genes chosen from the group consisting of NDUFA4l2, ANGPTL4, EGLN3, PTHLH, and ATP6V1B1 in a sample originating from said human individual;
b) establishing up, or down, regulation of expression of said one or more genes as compared to expression of said respective one or more genes in a sample originating from said human individual not comprising kidney tumour cells or tissue, or from an individual, or group of individuals, not suffering from kidney cancer; and
c) establishing the presence, or absence, of a kidney tumour based on the established up- or down regulation of said one or more genes.

According to the present invention establishing the presence, or absence, of a kidney, or renal, tumour in a human individual preferably comprises prognosis and/or prediction of disease survival.

It should be noted that the present method, taken alone, does not suffice to diagnose an individual as suffering from kidney cancer. For this, a trained physician is needed capable of taking into account factors not related to the present invention as disease symptoms, history, pathology, general condition, age, sex and/or other disease indicators. The present method provides the trained physician with an additional tool, or aid, to arrive at a reliable diagnosis.

According to the present invention, expression analysis comprises establishing an increased, or decreased, expression of a gene as compared to expression of the gene in non-kidney cancer tissue, i.e., under non-disease conditions. For example establishing an increased expression of NDUFA4L2, ANGPTL4, EGLN3, PTHLH and/or a decreased expression of ATP6V1B1 as compared to expression of these genes under non-kidney cancer conditions, allows establishing the presence, or absence, of a kidney, or renal, tumour in a human individual suspected of suffering from kidney, or renal, cancer.

NDUFA4L2: NADH Dehydrogenase is the first enzyme (complexI) of the mitochondrial electron transport chain. In this chain, the complex translocates 4 protons across the inner membrane per molecule of oxidised NADH, helping to build the electrochemical potential used to produce ATP. NADH Dehydrogenase is the largest of the respiratory complexes, the mammalian enzyme containing 45 separate polypeptide chains.

The catalytic properties of the complex are not simple. Two catalytically and structurally distinct forms exist: one is the so-called “active” A-form and the other is the catalytically silent “de-activated” D-form. These conformational differences have a very important physiological significance. It is likely that transition from the active to the de-active form takes place during pathological conditions, during hypoxia or when the tissue nitric oxide:oxygen ratio increases.

ANGPTL4: Angiopoietin-like 4 is a member of the angiopoietin/angiopoietin-like family and encodes a glycosylated, secreted protein with a fibrinogen C-terminal domain. This gene is induced under hypoxic conditions in endothelial cells and is a target of peroxisome proliferatin activators. The encoded protein may play a role in several cancers and it also has been shown to prevent the metastatic process by inhibiting vascular activity as well as tumour cell motility and invasiveness. The gene may act as a regulator of angiogenesis and modulate tumourgenesis. It inhibits proliferation, migration and reduces vascular leakage.

EGLN3: EGL nine homolog 3 catalyzes the post-translational formation of 4-hydroxyproline in hypoxia-inducible factor (HIF) alpha proteins. EGLN3 hydoxylates HIF-1 alpha at ‘Pro-564’. It functions as a cellular oxygen sensor and targets HIF through the hydroxylation for proteasomal degradation via the von Hippel-Lindau ubiquitination complex.

PTHLH: parathyroid hormone-like hormone. The protein encoded by this gene is a member of the parathyroid hormone family. This neuroendocrine peptide is a critical regulator of cellular and organ growth, development, migration, differentiation and survival and of epithelial calcium ion transport. It regulates endochondral bone development and epithelial-mesenchymal interactions during the formation of the mammary glands and teeth. The receptor of this hormone, PTHR1, is responsible for most cases of humoral hypercalcemia of malignancy.

ATP6V1B1: ATPase, H+ transporting, lysosomal 56/58 kDa, V1 subunit B1. This gene encodes a component of vacuolar ATPase (V-ATPase), a multisubunit enzyme that mediates acidification of eukaryotic intracellular organelles. V-ATPase dependent organelle acidification is necessary for such intracellular processes as protein sorting, receptor-mediated endocytosis, and synaptic vesicle proton gradient generation.

V-ATPase is composed of a cytosolic V1 domain and a transmembrane V0 domain. The V1 domain consists of three A and three B subunits, two G subunits plus the C, D, E, F, and H subunits. The V1 domain contains the ATP catalytic site. The V0 domain consists of five different subunits. Additional isoforms of many of the V1 and V0 subunit proteins are encoded by multiple genes or alternatively spliced transcript variants. This encoded protein is one of two V1 domain B subunit isoforms and is found in the kidney. Mutations in this gene cause distal renal tubular acidosis associated with sensorineural deafness.

According to the present invention, the method as described above is preferably an ex vivo and/or in vitro method. In this embodiment, expression analysis of the indicated genes is performed on a sample derived, originating or obtained from the individual suspected of suffering from kidney, or renal, cancer. Such sample can be a bodily fluid such as saliva, lymph, blood or urine, or a tissue sample such as a renal biopsy. Samples of, derived or originating from blood and urine are preferably contemplated within the context of the present invention as are samples of, derived or originating from renal biopsies.

According to another preferred embodiment of the present method, determining the expression comprises determining mRNA expression of said one or more genes.

Expression analysis based on mRNA is generally known in the art and routinely practiced in diagnostic labs world-wide. For example, suitable techniques for mRNA analysis are Northern blot hybridisation and amplification based techniques such as PCR, and especially real time PCR, and NASBA.

According to a particularly preferred embodiment, expression analysis comprises high-throughput DNA array chip analysis not only allowing the simultaneous analysis of multiple samples but also automatic analysis processing.

According to another preferred embodiment of the present method, determining the expression comprises determining protein levels of the genes. Suitable techniques are, for example, matrix-assisted laser desorption-ionization time-of-flight mass spectrometer (MALDI-TOF).

According to the present invention, the present method is preferably provided by expression analysis of two or more, preferably three or more, more preferably four or more, most preferably five of the genes chosen from the group consisting of NDUFA4L2, ANGPTL4, EGLN3, PTHLH and/or ATP6V1B1.

According to a particularly preferred embodiment, the present method of diagnosis is provided by expression analysis of NDUFA4L2, ANGPTL4, EGLN3, PTHLH and/or ATP6V1B1.

According to a most preferred embodiment of the above method, the present invention relates to methods, wherein establishing the presence, or absence, of a tumour further comprises establishing metastasis or no metastasis. Establishing whether the kidney tumour identified is capable to metastasize, or has metastasized, is inherently a valuable tool for a trained physician to develop an individualised treatment protocol. In case of metastasis, the survival rate of a patient is generally directly correlated with the point in time on which the metastasis is identified, detected or established. The earlier in time the treatment commences, the higher the survival rates. Additionally, if a tumour is not capable of metastasis, is not likely to metastasize, or has not metastasized, the patient needs not to be subjected to, or can be spared of, treatments severely affecting the quality of life.

The kidney, or renal tumour, identified with the present methods is preferably a renal cell carcinoma or RCC.

Considering the provided diagnostic value for a trained physician of the present genes as biomarkers for kidney cancer, the present invention also relates to the use of expression analysis of one or more genes selected from the group consisting of NDUFA4L2, ANGPTL4, EGLN3, PTHLH and/or ATP6V1B1 for establishing the presence, or absence, of a kidney tumour in a human individual suspected of suffering from kidney cancer.

The present use, for reasons indicated above, is preferably an ex vivo or in vitro use and, preferably, involves the use of two or more, three or more, four or more, and five for establishing the presence, or absence, of a kidney tumour in a human individual suspected of suffering from kidney cancer.

The kidney, or renal tumour, identified using the present genes is preferably a renal cell carcinoma or RCC

Again, considering the diagnostic value for a trained physician of the present genes as biomarkers for kidney, or renal cancer, the present invention also relates to a kit of parts for establishing the presence, or absence, of a kidney tumour in a human individual suspected of suffering from kidney cancer, said kit of parts comprises:
expression analysis means for determining the expression of one or more genes one or more genes chosen from the group consisting of NDUFA4l2, ANGPTL4, EGLN3, PTHLH, and ATP6V1B1;
instructions for use.

According to a preferred embodiment, the present kit of parts comprises mRNA expression analysis means, preferably for PCR, rtPCR or NASBA.

According to a particularly preferred embodiment, the present kit of parts comprises means for expression analysis of two or more, three or more, four or more or five of the present genes.

In the present description, reference is made to genes suitable as bio- or molecular markers for kidney cancer by referring to their arbitrarily assigned names. Although the skilled person is readily capable of identifying and using the present genes based on the indicated names, the appended figures provide the cDNA sequence of these genes, thereby allowing the skilled person to develop expression analysis assays based on analysis techniques commonly known in the art.

Such analysis techniques can, for example, be based on the genomic sequence of the gene or the provided cDNA or amino acid sequences. This sequence information can either be derived from the provided sequences, or can be readily obtained from public databases, for example by using the provided accession numbers.

The present invention will be further elucidated in the following examples of preferred embodiments of the invention. In the examples, reference is made to figures, wherein:

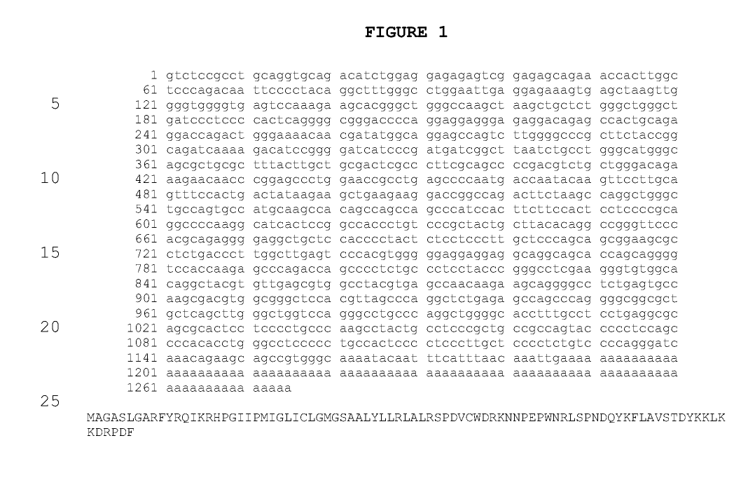

FIGS. 1-5: show the cDNA and amino acid sequences of the NDUFA4L2 gene (NM_{—}020142, NP_{—}064527); the ANGPTL4 gene (NM_{—}139314, NP_{—}647475); the EGLN3 gene (NM_{—}022073, NP_{—}071356); the PTHLH gene (NM_{—}198965, NP_{—}945316); and the ATP6V1B1 gene (NM_{—}001692, NP_{—}001683), respectively;

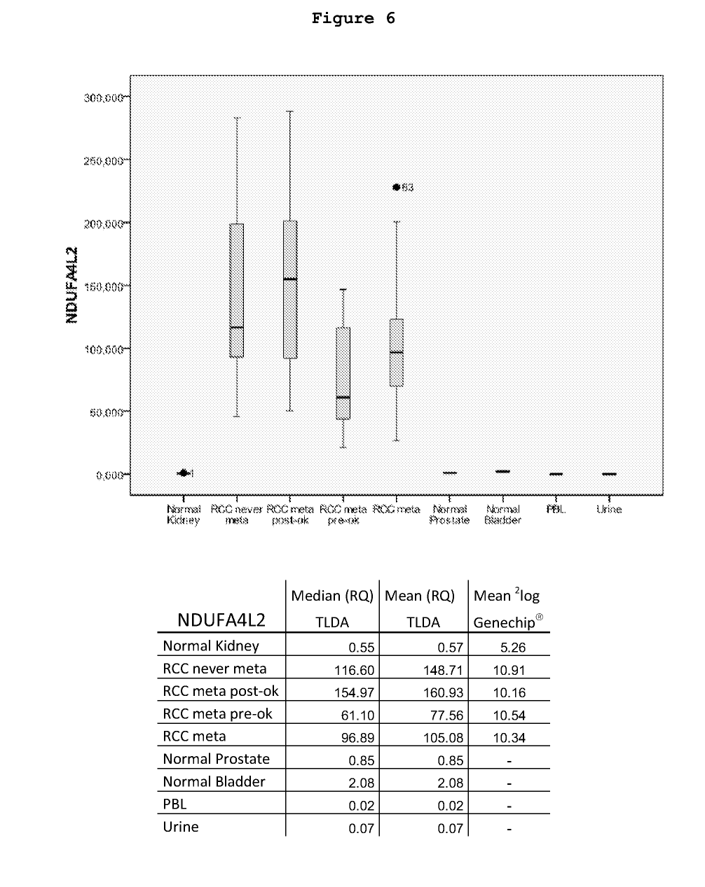

FIGS. 6-10: show boxplot TLDA data based on the groups normal kidney, RCC specimens from patients that never showed metastasis, RCC specimens from patients showing metastasis after nephrectomy, RCC specimens from patients showing metastasis before their nephrectomy and metastasis from RCC patients.

### EXAMPLE 1

To identify markers for kidney cancer, the gene expression profile (GeneChip® Human Exon1.0 ST Array, Affymetrix) of samples from patients with and without kidney cancer were used.

The expression analysis is performed according to standard protocols. Briefly, tissue was obtained after radical nephrectomy from patients with kidney cancer. The tissues were snap frozen and cryostat sections were H.E. stained for classification by a pathologist.

Malignant- and non-malignant areas were dissected and total RNA was extracted with TRIzol (Invitrogen, Carlsbad, Calif., USA) following manufacturer's instructions. The total RNA was purified with the Qiagen RNeasy mini kit (Qiagen, Valencia, Calif., USA). Integrity of the RNA was checked by electrophoresis using the Agilent 2100 Bioanalyzer.

From the purified total RNA, 1 μg was used for the GeneChip® Whole Transcript (WT) Sense Target Labeling Assay. (Affymetrix, Santa Clara, Calif., USA). According to the protocol of this assay, the majority of ribosomal RNA was removed using a RiboMinus Human/Mouse Transcriptome Isolation Kit (Invitrogen, Carlsbad, Calif., USA). Using a random hexamer incorporating a T7 promoter, double-stranded cDNA was synthesized. Then cRNA, was generated from the double-stranded cDNA template through an in vitro transcription reaction and purified using the Affymetrix sample clean-up module. Single-stranded cDNA was regenerated through a random-primed reverse transcription using a dNTP mix containing dUTP. The RNA was hydrolyzed with RNaseH and the cDNA was purified. The cDNA was then fragmented by incubation with a mixture of UDG (uracil DNA glycosylase) and APE1 (apurinic/apyrimidinic endonuclease 1) restriction endonucleases and, finally, end-labeled via a terminal transferase reaction incorporating a biotinylated dideoxynucleotide.

Of the fragmented, biotinylated cDNA, 5.5 μg was added to a hybridization mixture, loaded on a Human Exon1.0 ST GeneChip® and hybridized for 16 hours at 45° C. and 60 rpm.

Using the Affymetrix exon array, genes are indirectly measured by exons analysis which measurements can be combined into transcript clusters measurements. There are more than 300,000 transcript clusters on the array, of which 90,000 contain more than one exon. Of these 90,000 there are more than 17,000 high confidence (CORE) genes which are used in the default analysis. In total there are more than 5.5 million features per array.

Following hybridization, the array was washed and stained according to the Affymetrix protocol. The stained array was scanned at 532 nm using an Affymetrix GeneChip® Scanner 3000, generating CEL files for each array.

Exon-level expression values were derived from the CEL file probe-level hybridization intensities using the model-based RMA algorithm as implemented in the Affymetrix Expression Console™ software. RMA (Robust Multiarray Average) performs normalization, background correction and data summarization. Differentially expressed genes between conditions are calculated using Anova (ANalysis Of Variance), a T-test for more than two groups.

The target identification is biased since clinically well defined risk groups were analyzed. The markers are categorized based on their role in cancer biology. For the identification of markers the RCC group is compared with normal kidney group.

Based on the expression analysis obtained, biomarkers were identified based on 3 RCC and 3 normal kidney specimens. The expression profiles of the biomarkers are provided in Table 1.

TABLE 1
Expression characteristics of 20 targets characterizing RCC, based on the analysis of 3 well
annotated RCC and 3 normal kidney specimens.

| | Gene | Expression | Fold | |
|---|---|---|---|---|
| Gene name | Assignment | in RCC | Change | Rank |
| parathyroid hormone-like hormone | NM_198965 | up | 446 | 1 |
| (PTHLH) | | | | |
| neuronal pentraxin II (NPTX2) | NM_002523 | up | 290 | 2 |
| NADH dehydrogenase 1 alpha | NM_020142 | up | 110 | 5 |
| subcomplex, 4-like 2 (NDUFA4L2) | | | | |
| angiopoietin like 4 (ANGPTL4) | NM_139314 | up | 68 | 8 |
| hexokinase 2 (HK2) | NM_000189 | up | 61 | 9 |
| egl nine homolog 3 (EGLN3) | NM_022073 | up | 20 | 19 |
| insulin-like growth factor binding protein 3 | NM_001013398 | up | 19 | 21 |
| (IGFBP3) | | | | |
| lysyl oxidase (LOX) | NM_002317 | up | 19 | 22 |
| lipoma HMGIC fusion partner-like 2 | NM_005779 | up | 19 | 23 |
| (LHFPL2) | | | | |
| solute carrier family 2 member 1 (SLC2A1) | NM_006516 | up | 18 | 25 |
| solute carrier family 12 member 3 | NM_000339 | down | 239.2 | 1 |
| (SLC12A3) | | | | |
| secreted frizzled-related protein 1 (SFRP1) | NM_003012 | down | 163.4 | 2 |
| claudin 8 (CLDN8) | NM_199328 | down | 62.7 | 4 |
| crystallin, alpha A (CRYAA) | NM_000394 | down | 43.9 | 8 |
| parathyroid hormone receptor 1 (PTHR1) | NM_000316 | down | 40.5 | 11 |
| ATPase, H+ transporting, lysosomal 56/58 kDa, | NM_001692 | down | 39.9 | 12 |
| subunit B1 (ATP6B1) | | | | |
| X-prolyl aminopeptidase 2, (XPNPEP2) | NM_003399 | down | 35.2 | 15 |
| dipeptidase 1 (renal) (DPEP1) mRNA | NM_004413 | down | 33.2 | 16 |
| transcription factor CP2-like 1 (TFCP2L1) | NM_014553 | down | 30.9 | 18 |
| kininogen (KNG1) | NM_000893 | down | 25.0 | 21 |

### EXAMPLE 2

The protocol of example 1 was repeated on a group of 28 specimens; 18 well annotated RCC and 10 normal kidney samples.

The results obtained are presented in Table 2.

TABLE 2
Expression characteristics of 20 targets validated in the panel of 18 well annotated RCC and
10 normal kidney specimens.

| | Gene | Expression | Fold | |
|---|---|---|---|---|
| Gene name | Assignment | in RCC | Change | Rank |
| parathyroid hormone-like hormone | NM_198965 | up | 5.2 | 93 |
| (PTHLH) | | | | |
| neuronal pentraxin II (NPTX2) | NM_002523 | up | 7.7 | 38 |
| NADH dehydrogenase 1 alpha | NM_020142 | up | 38.6 | 2 |
| subcomplex, 4-like 2 (NDUFA4L2) | | | | |
| angiopoietin like 4 (ANGPTL4) | NM_139314 | up | 16.4 | 8 |
| hexokinase 2 (HK2) | NM_000189 | up | 9.5 | 24 |
| egl nine homolog 3 (EGLN3) | NM_022073 | up | 14.5 | 12 |
| insulin-like growth factor binding protein 3 | NM_001013398 | up | 9.0 | 27 |
| (IGFBP3) | | | | |
| lysyl oxidase (LOX) | NM_002317 | up | 11.8 | 18 |
| lipoma HMGIC fusion partner-like 2 | NM_005779 | up | 5.5 | 80 |
| (LHFPL2) | | | | |
| solute carrier family 2 member 1 (SLC2A1) | NM_006516 | up | 5.7 | 72 |
| solute carrier family 12 member 3 | NM_000339 | down | 44.4 | 19 |
| (SLC12A3) | | | | |
| secreted frizzled-related protein 1 (SFRP1) | NM_003012 | down | 17.2 | 72 |
| claudin 8 (CLDN8) | NM_199328 | down | 27.9 | 39 |
| crystallin, alpha A (CRYAA) | NM_000394 | down | 13.9 | 88 |
| parathyroid hormone receptor 1 (PTHR1) | NM_000316 | down | 12.9 | 97 |
| ATPase, H+ transporting, lysosomal 56/58 kDa, | NM_001692 | down | 16.6 | 74 |
| subunit B1 (ATP6B1) | | | | |
| X-prolyl aminopeptidase 2, (XPNPEP2) | NM_003399 | down | 46.7 | 17 |
| dipeptidase 1 (renal) (DPEP1) mRNA | NM_004413 | down | 36.0 | 30 |
| transcription factor CP2-like 1 (TFCP2L1) | NM_014553 | down | 26.9 | 42 |
| kininogen (KNG1) | NM_000893 | down | 187.7 | 4 |

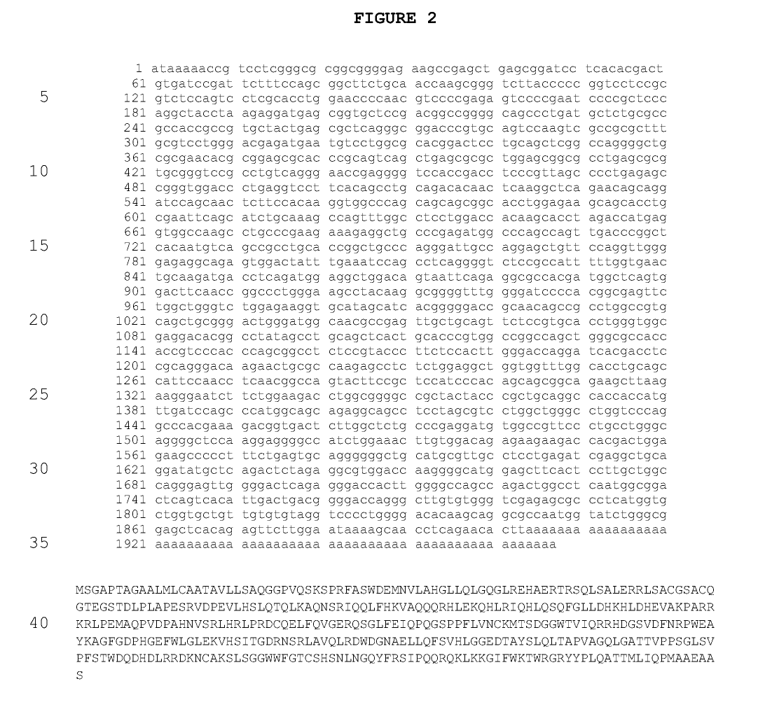

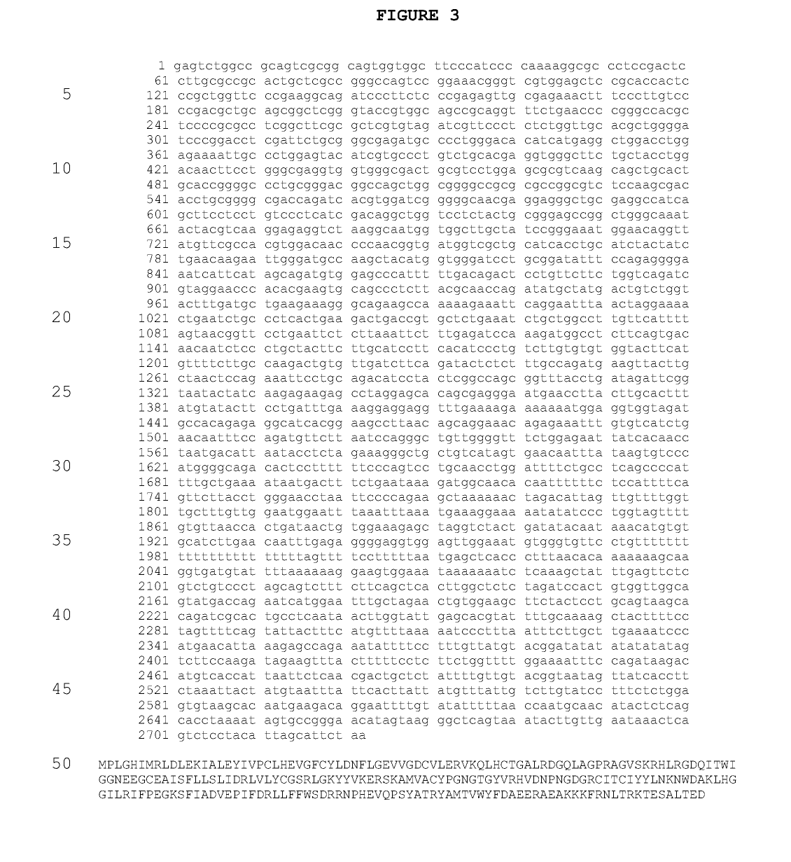

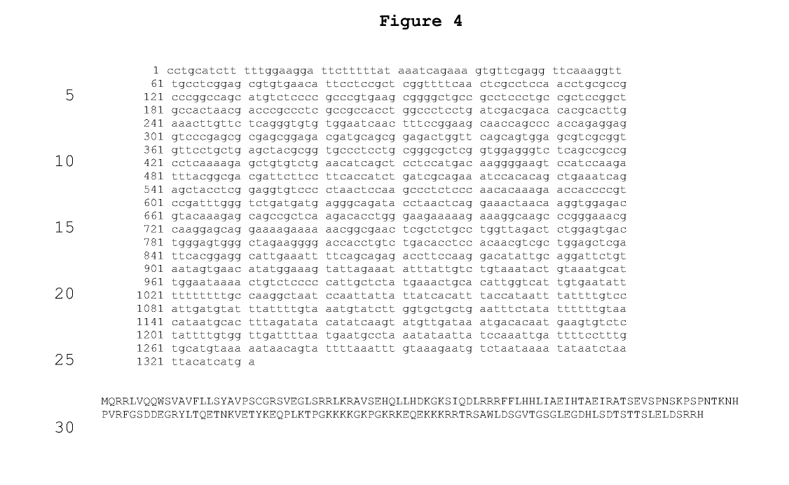

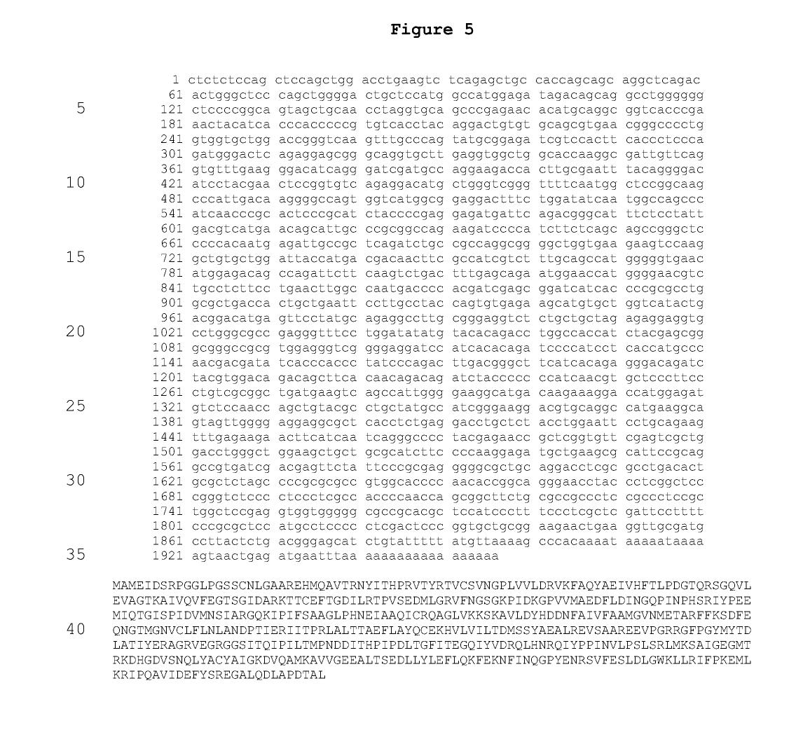

As can be clearly seen in tables 1 and 2, an up regulation of expression of PTHLH (FIG. 4), NPTX2, NDUFA4L2 (FIG. 1), ANGPTL4 (FIG. 2), HK2, EGLN3 (FIG. 3), IGFBP3, LOX, LHFPL2 and SLC2A1 was associated with kidney cancer. Further, as can be clearly seen in tables 1 and 2, a down-regulation of expression of SLC12A3, SFRP1, CLDN8, CRYAA, PTHR1, ATP6V1B1 (FIG. 5), XPNPEP2, DPEP1, TFCP2L1 and KNG1 was associated with kidney cancer.

Considering the above results obtained in 28 samples, the expression data clearly demonstrate the suitable of these genes as biomarkers for the diagnosis of kidney cancer.

### EXAMPLE 3

The group of 28 specimens of example 2 was expanded with 44 specimens. Enlargement of the number of specimen enabled analyzing subgroups of patients with RCC. These subgroups were based on aggressiveness and the total number of 72 specimens could be sub-divided into the following groups: Normal kidney (n=21), RCC specimens that never showed metastasis (n=14), RCC specimens from patients showing metastasis after nephrectomy (n=12), RCC specimens from patients showing metastasis before their nephrectomy (n=14) and metastasis from RCC patients (n=11). The results obtained are presented in Table 3.

TABLE 3
Expression characteristics of 20 targets validated in the panel of 40 well annotated RCC,
11 kidney cancer metastasis and 21 normal kidney specimens. Values in ²log scale.

| | Expression | group1 | group 2 | group 3 | group 4 | group 5 |
|---|---|---|---|---|---|---|
| Gene name | in NK | NK | never | post | pre | meta |
| parathyroid hormone-like | up | 3.84 | 5.26 | 6.63 | 7.23 | 5.62 |
| hormone (PTHLH) | | | | | | |
| neuronal pentraxin II | up | 4.07 | 7.44 | 6.84 | 8.11 | 7.76 |
| (NPTX2) | | | | | | |
| NADH dehydrogenase 1 | up | 5.26 | 10.91 | 10.16 | 10.54 | 10.34 |
| alpha subcomplex, 4-like 2 | | | | | | |
| (NDUFA4L2) | | | | | | |
| angiopoietin like 4 | up | 3.92 | 8.83 | 7.86 | 8.17 | 7.66 |
| (ANGPTL4) | | | | | | |
| hexokinase 2 (HK2) | up | 4.30 | 7.24 | 7.71 | 7.20 | 7.03 |
| egl nine homolog 3 (EGLN3) | up | 5.50 | 9.71 | 9.3 | 9.36 | 9.23 |
| insulin-like growth factor | up | 7.18 | 10.65 | 10.31 | 10.35 | 9.76 |
| binding protein 3 (IGFBP3) | | | | | | |
| lysyl oxidase (LOX) | up | 6.24 | 9.61 | 9.72 | 10.40 | 9.65 |
| lipoma HMGIC fusion | up | 5.15 | 7.34 | 7.83 | 7.94 | 7.95 |
| partner-like 2 (LHFPL2) | | | | | | |
| solute carrier family 2 | up | 7.26 | 9.66 | 9.61 | 9.89 | 9.19 |
| member 1 (SLC2A1) | | | | | | |
| solute carrier family 12 | down | 9.62 | 4.19 | 4.34 | 4.36 | 4.30 |
| member 3 (SLC12A3) | | | | | | |
| secreted frizzled-related | down | 8.05 | 3.65 | 3.78 | 3.79 | 3.81 |
| protein 1 (SFRP1) | | | | | | |
| claudin 8 (CLDN8) | down | 8.39 | 3.66 | 3.77 | 3.62 | 3.63 |
| crystallin, alpha A (CRYAA) | down | 8.82 | 4.88 | 5.01 | 5.16 | 5.09 |
| parathyroid hormone | down | 8.54 | 5.03 | 4.73 | 4.56 | 4.26 |
| receptor 1 (PTHR1) | | | | | | |
| ATPase, H+ transporting, | down | 8.40 | 4.36 | 4.44 | 4.42 | 4.36 |
| lysosomal subunit B1 | | | | | | |
| (ATP6B1) | | | | | | |
| X-prolyl aminopeptidase | down | 8.99 | 3.79 | 3.80 | 3.61 | 3.59 |
| 2, (XPNPEP2) | | | | | | |
| dipeptidase 1 (renal) | down | 9.86 | 4.95 | 4.92 | 4.77 | 4.68 |
| (DPEP1) | | | | | | |
| transcription factor CP2-like | down | 9.22 | 4.25 | 5.2 | 4.85 | 4.77 |
| 1 (TFCP2L1) | | | | | | |
| kininogen (KNG1) | down | 10.25 | 3.03 | 3.26 | 3.23 | 3.16 |

### EXAMPLE 4

Using the gene expression profile (GeneChip® Human Exon1.0 ST Array, Affymetrix) on 72 tissue specimens of normal kidney, kidney cancer (RCC) and kidney cancer metastasis, several genes were found to be differentially expressed. Together with several other in the GeneChip® Human Exon1.0 ST Array differentially expressed genes and some housekeeping and reference genes (HPRT1, GAPDH, B2m, TBP, PPIA), the expression levels of these genes were validated using the TaqMan® Low Density arrays (TLDA, Applied Biosystems). In Table 4 an overview of the validated genes is shown.

TABLE 4
Gene expression assays used for TLDA analysis

| | | | Applied |
|---|---|---|---|
| | | Accesion | Biosystems |
| Symbol | Gene description | number | Taqman assay nr. |
| NDUFA4L2 | NADH dehydrogenase 1 alpha | NM_020142 | Hs00220041_m1 |
| | subcomplex, 4-like 2 | | |
| ANGPTL4 | angiopoietin-like 4 | NM_139314 | Hs01101127_m1 |
| EGLN3 | egl nine homolog 3 | NM_022073 | Hs00222966_m1 |
| LOX | lysyl oxidase | NM_002317 | Hs00942480_m1 |
| NPTX2 | neuronal pentraxin II | NM_002523 | Hs00383983_m1 |
| IGFBP3 | insulin-like growth factor binding protein 3 | NM_001013398 | Hs00426289_m1 |
| HK2 | hexokinase 2 | NM_000189 | Hs00606086_m1 |
| LHFPL2 | lipoma HMGIC fusion partner-like 2 | NM_005779 | Hs00299613_m1 |
| PTHLH | parathyroid hormone-like hormone | NM_198965 | Hs00174969_m1 |
| SLC2A1) | solute carrier family 2, member 1 | NM_006516 | Hs00892681_m1 |
| KNG1 | kininogen 1 | NM_000893 | Hs00949376_m1 |
| XPNPEP2 | X-prolyl aminopeptidase 2, membrane-b | NM_003399 | Hs00950918_m1 |
| SLC12A3 | solute carrier family 12, member 3 | NM_000339 | Hs01027568_m1 |
| DPEP1 | dipeptidase 1 (renal) | NM_004413 | Hs01116752_m1 |
| CLDN8 | claudin 8 | NM_199328 | Hs00273282_s1 |
| TFCP2L1 | transcription factor CP2-like 1 | NM_014553 | Hs00232708_m1 |
| SFRP1 | secreted frizzled-related protein 1 | NM_003012 | Hs00610060_m1 |
| PTH1R | parathyroid hormone 1 receptor | NM_000316 | Hs00174895_m1 |
| ATP6V1B1 | ATPase, H+ transporting, lysosomal, | NM_001692 | Hs00266092_m1 |
| | V1 subunit | | |
| CRYAA | crystallin, alpha A | NM_000394 | Hs00166138_m1 |
| HIG2 | hypoxia-inducible protein 2 | NM_013332 | Hs00203383_m1 |
| ENO2 | enolase 2 (gamma, neuronal) | NM_001975 | Hs00157360_m1 |
| RRM2 | ribonucleotide reductase M2 | NM_001034 | Hs00357247_g1 |
| | polypeptide | | |
| NETO2 | neuropilin (NRP) and tolloid (TLL)-like 2 | NM_018092 | Hs00983152_m1 |
| BHLHE41 | basic helix-loop-helix family, member | NM_030762 | Hs00229146_m1 |
| | e41 | | |
| RGS1 | regulator of G-protein signaling 1 | NM_002922 | Hs00175260_m1 |
| FCGR3A | Fc fragment of IgG, low affinity IIIa | NM_000569 | Hs02388314_m1 |
| SLC16A3 | solute carrier family 16, member 3 | NM_001042422 | Hs01108070_m1 |
| PDK1 | pyruvate dehydrogenase kinase, | NM_002610 | Hs00176853_m1 |
| | isozyme 1 | | |
| PCSK6 | proprotein convertase subtilisin/kexin | NM_002570 | Hs00159844_m1 |
| | type 6 | | |
| TGFB1 | transforming growth factor, beta 1 | NM_000660 | Hs00998130_m1 |
| SLC12A1 | solute carrier family 12, member 1 | NM_000338 | Hs00165731_m1 |
| UMOD | uromodulin | NM_003361 | Hs00358451_m1 |
| C12orf59 | chromosome 12 open reading frame 59 | NM_153022 | Hs00541808_m1 |
| C4orf31 | chromosome 4 open reading frame 31 | NM_024574 | Hs00375503_m1 |
| SPINK1 | serine peptidase inhibitor, Kazal type 1 | NM_003122 | Hs00162154_m1 |
| PRRX1 | paired related homeobox 1 | NM_006902 | Hs00246569_m1 |
| PCDHB3 | protocadherin beta 3 | NM_018937 | Hs00251772_s1 |
| PCDH21 | protocadherin 21 | NM_033100 | Hs00326445_m1 |
| PRAME | PRAME, preferentially expressed | NM_206953 | Hs01022301_m1 |
| | antigen in melanoma | | |
| IL20RB | interleukin 20 receptor beta | NM_144717 | Hs00376373_m1 |
| IL8 | interleukin 8 | NM_000584 | Hs99999034_m1 |
| CP | ceruloplasmin (ferroxidase) | NM_000096 | Hs00236810_m1 |
| TBP | TATA box binding protein | NM_003194 | Hs00920498_m1 |
| PPIA | peptidylprolyl isomerase A (cyclophilin A) | NM_021130 | Hs99999904_m1 |
| B2M | beta-2-microglobulin | NM_004048 | Hs00187842_m1 |
| HPRT1 | hypoxanthine | NM_000194 | Hs01003267_m1 |
| | phosphoribosyltransferase 1 | | |
| GAPDH | GAPDH | NM_002046 | Hs99999905_m1 |

The validation with TLDA analysis was performed with 69 kidney samples. Among these 41 samples were newly selected/isolated, 28 samples were previously used in the identification with the GeneChip® Human Exon1.0 ST Array. Kidney cancer specimens in the following categories were used (see also Table 5 below): Normal kidney (n=16), RCC specimens that never showed metastasis (n=11), RCC specimens from patients showing metastasis after nephrectomy (n=18), RCC specimens from patients showing metastasis before their nephrectomy (n=11) and metastasis from RCC patients (n=13).

To determine whether the identified biomarkers for RCC could be used in a to be established kit for specific detection in body fluids like urine and blood, the expression levels of these markers in a number of reference samples was determined. These samples included normal bladder tissue (n=2), normal prostate tissue (n=2), peripheral blood lymphocytes from healthy individuals (n=4) and urine samples from normal patients (n=2). For positive correlation of the specific detection of an identified biomarker and the presence of kidney cancer the expression of this biomarker should ideally be low in these normal reference samples.

All tissue samples were snap frozen and cryostat sections were stained with hematoxylin and eosin (H.E.). These H.E.-stained sections were classified by a pathologist. Tumor areas were dissected. RNA was extracted from 10 μm thick serial sections that were collected from each tissue specimen at several levels. Tissue was evaluated by HE-staining of sections at each level and verified microscopically. Total RNA was extracted with TRIzol® (Invitrogen, Carlsbad, Calif., USA) according to the manufacturer's instructions. Total RNA was purified using the RNeasy mini kit (Qiagen, Valencia, Calif., USA). RNA quantity and quality were assessed on a NanoDrop 1000 spectrophotometer (NanoDrop Technologies, Wilmington, Del., USA) and on an Agilent 2100 Bioanalyzer (Agilent Technologies Inc., Santa Clara, Calif., USA).

Two μg DNase-treated total RNA was reverse transcribed using SuperScript™ II Reverse Transcriptase (Invitrogen) in a 37.5 μl reaction according to the manufacturer's protocol. Reactions were incubated for 10 minutes at 25° C., 60 minutes at 42° C. and 15 minutes at 70° C. To the cDNA, 62.5 μl milliQ was added.

Gene expression levels were measured using the TaqMan® Low Density Arrays (TLDA; Applied Biosystems). A list of assays used in this study is given in Table 4. Of the individual cDNAs, 3 μl is added to 50 μl Taqman® Universal Probe Master Mix (Applied Biosystems) and 47 μl milliQ. One hundred μl of each sample was loaded into 1 sample reservoir of a TaqMan® Array (384-Well Micro Fluidic Card) (Applied Biosystems). The TaqMan® Array was centrifuged twice for 1 minute at 280g and sealed to prevent well-to-well contamination. The cards were placed in the micro-fluid card sample block of an 7900 HT Fast Real-Time PCR System (Applied Biosystems). The thermal cycle conditions were: 2 minutes 50° C., 10 minutes at 94.5° C., followed by 40 cycles for 30 seconds at 97° C. and 1 minute at 59.7° C.

Raw data were recorded with the Sequence detection System (SDS) software of the instruments. Micro Fluidic Cards were analyzed with RQ documents and the RQ Manager Software for automated data analysis. Delta cycle threshold (Ct) values were determined as the difference between the Ct of each test gene and the Ct of hypoxanthine phosphoribosyltransferase 1 (HPRT) (endogenous control gene). Furthermore, gene expression values were calculated based on the comparative threshold cycle (Ct) method, in which a normal kidney RNA sample was designated as a calibrator to which the other samples were compared.

For the validation of the differentially expressed genes found by the GeneChip® Human Exon1.0 ST Array, 69 kidney specimens were used in TaqMan® Low Density arrays (TLDAs). In these TLDAs, expression levels were determined for the 48 genes of interest. The kidney tissue specimens were put in order from normal kidney, RCC specimens from patients who never showed metastasis, RCC specimens from patients showing metastasis after nephrectomy, RCC specimens from patients showing metastasis before their nephrectomy and finally to metastasis from RCC patients. Both GeneChip® Human Exon1.0 ST Array and TLDA data were analyzed using scatter- and box plots.

From the expression levels of the genes scatter- and boxplots were made in which the kidney specimens were put in order from normal kidney, RCC specimens from patients who never showed metastasis, RCC specimens from patients showing metastasis after nephrectomy, RCC specimens from patients showing metastasis before their nephrectomy and finally to metastasis from RCC patients.

In the same plots the (background) expression levels of the genes in normal prostate tissue, normal bladder tissue, blood (PBL) and urine were shown.

After analysis of the box- and scatterplots (FIGS. 5 to 10) a list of genes was derived the expression of which is indicative for establishing the presence, or absence, of a kidney tumour in a human individual suspected of suffering from kidney cancer comprising and, accordingly, indicative for kidney cancer and prognosis thereof.

TABLE 5
List of genes identified

| | | | Applied Biosystems |
|---|---|---|---|
| Symbol | Gene description | Figures | Taqman assay nr. |
| NDUFA4L2 | NADH dehydrogenase | 1.6 | Hs00220041_m1 |
| | 1 alpha subcomplex, | | |
| | 4-like 2 | | |
| ANGPTL4 | angiopoietin-like 4 | 2.7 | Hs01101127_m1 |
| EGLN3 | egl nine homolog 3 | 3.8 | Hs00222966_m1 |
| PTHLH | parathyroid hormone-like | 4.9 | Hs00174969_m1 |
| | hormone | | |
| ATP6V1B1 | ATPase, H+ transporting, | 5.10 | Hs00266092_m1 |
| | lysosomal, V1 subunit B1 | | |

NDUFA4L2 (FIG. 6): The present GeneChip® Human Exon1.0 ST Array data showed that NDUFA4L2 was highly up-regulated in renal cell carcinomas and RCC metastases. The expression of NDUFA4L2 within either of these three groups was highly constant. Validation experiments using TaqMan Low Density Arrays confirmed this high upregulation. Between the normal kidney and the RCC group a more than 100 fold difference in expression level was observed.

One of the criteria used in the selection procedure for biomarkers was that the selected gene should have a low expression in normal prostate, normal bladder, urine and PBL from healthy persons. NDUFA4L2 meets this criterion.

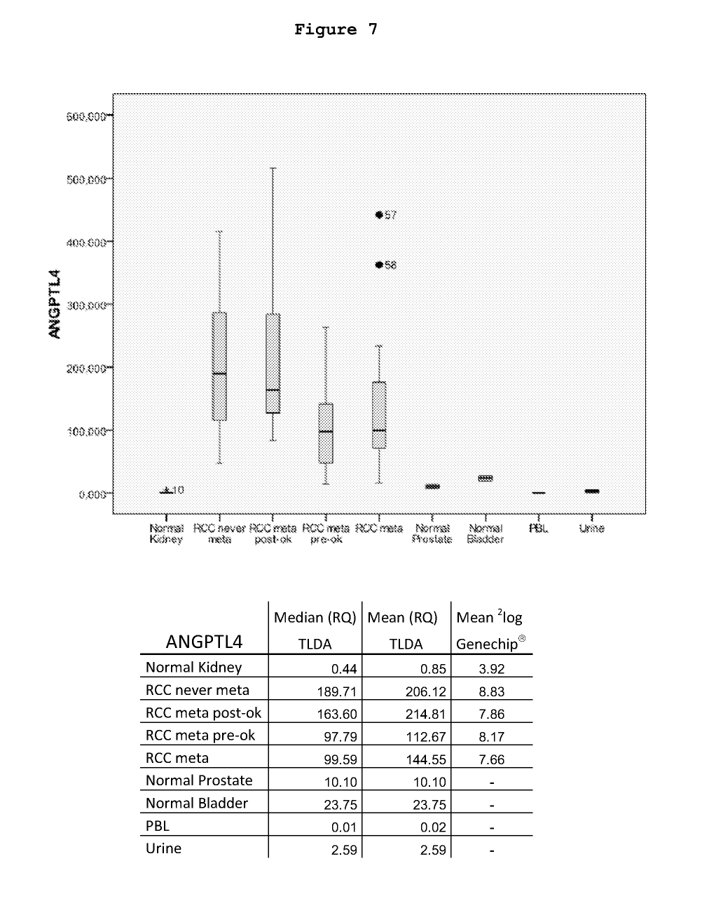

ANGPTL4 (FIG. 7): The present GeneChip® Human Exon1.0 ST Array data showed that ANGPTL4 was highly up-regulated in renal cell carcinomas and RCC metastases. The expression of NDUFA4L2 within either of these three groups was constant. Validation experiments using TaqMan Low Density Arrays confirmed this high upregulation. Between the normal kidney and the RCC group an almost more than 200 fold difference in expression level was observed.

One of the criteria used in the selection procedure for biomarkers was that the selected gene should have a low expression in normal prostate, normal bladder, urine and PBL from healthy persons. ANGPTL4 meets this criterion.

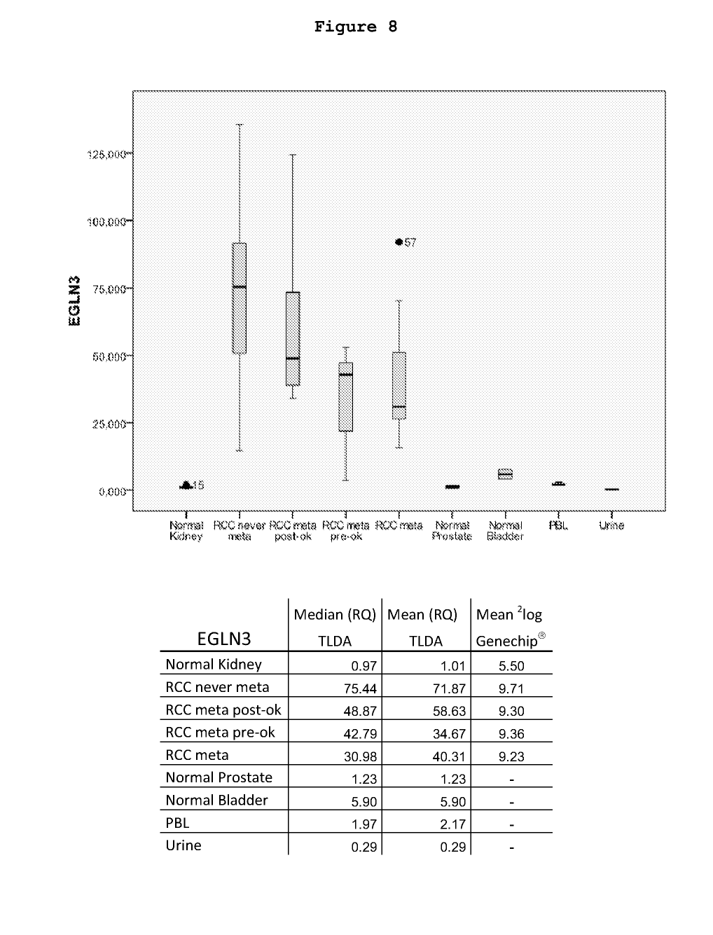

EGLN3 (FIG. 8): The present GeneChip® Human Exon 1.0 ST Array data showed that EGLN3 was highly up-regulated in renal cell carcinomas and RCC metastases. The expression of EGLN3 within either of these three groups was highly constant. Validation experiments using TaqMan Low Density Arrays confirmed this high upregulation. Between the normal kidney and the RCC group a 50 fold difference in expression level was observed.

One of the criteria used in the selection procedure for biomarkers was that the selected gene should have a low expression in normal prostate, normal bladder, urine and PBL from healthy persons. EGLN3 meets this criterion.

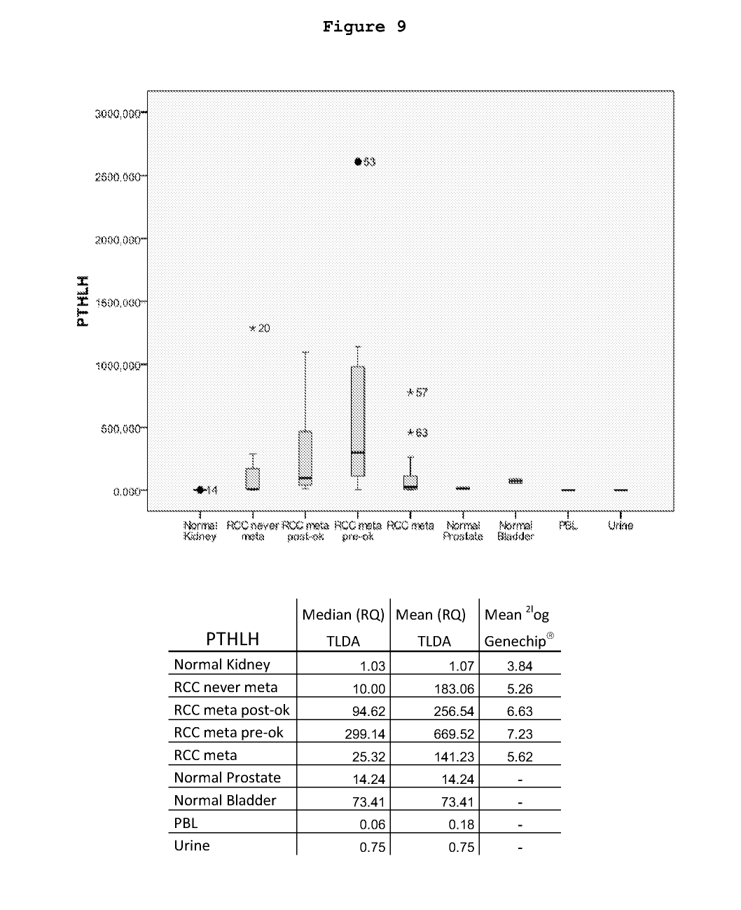

PTHLH (FIG. 9): The present GeneChip® Human Exon 1.0 ST Array data showed that PTHLH was up-regulated in renal cell carcinomas and RCC metastases. Validation experiments using TaqMan Low Density Arrays confirmed this upregulation. Between the normal kidney and the RCC group a more than 250 fold difference in expression level was observed.

Due to the high expression in the group “RCC meta pre-ok” this biomarker could be used for the identification of metastasis in patients who are scheduled for a nephrectomy.

One of the criteria used in the selection procedure for biomarkers was that the selected gene should have a low expression in normal prostate, normal bladder, urine and PBL from healthy persons. PTHLH meets this criterion.

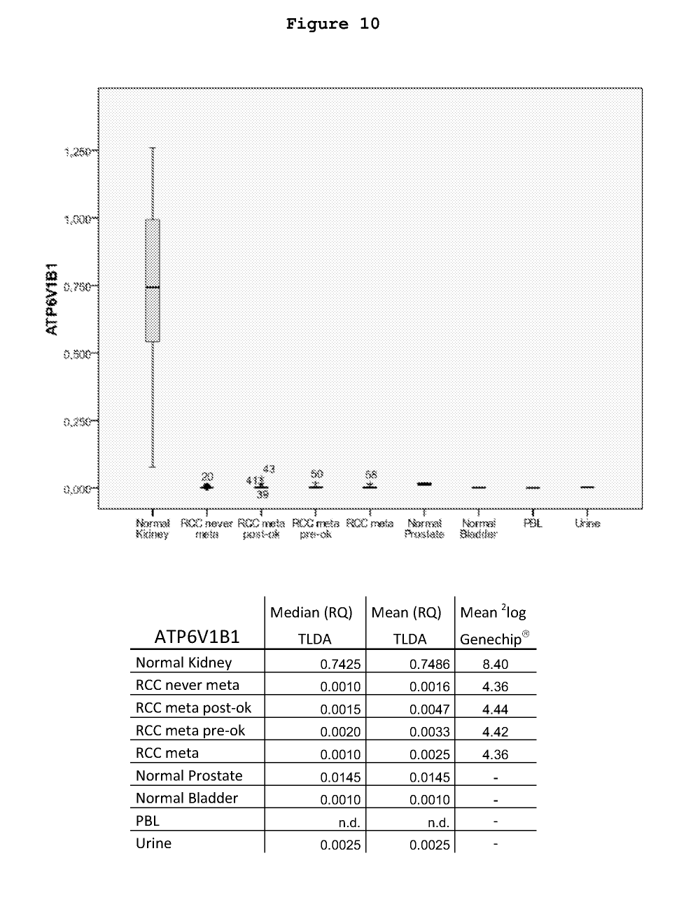

ATP6V1B1(FIG. 10): The present GeneChip® Human Exon1.0 ST Array data showed that ATP6V1B1 was strongly down-regulated in renal cell carcinomas and RCC metastases. The expression of ATP6V1B1 within either of these three groups was highly constant. Validation experiments using TaqMan Low Density Arrays confirmed this strong downregulation. Between the normal kidney and the RCC group a 250 fold difference in expression level was observed.

1.-15. (canceled)

16. Method for establishing the presence, or absence, of a kidney tumour in a human individual suspected of suffering from kidney cancer comprising:
a) determining the expression of one or more genes chosen from the group consisting of NDUFA4l2, ANGPTL4, EGLN3, PTHLH, and ATP6V1B1 in a sample originating from said human individual;
b) establishing up, or down, regulation of expression of said one or more genes as compared to expression of said respective one or more genes in a sample originating from said human individual not comprising kidney tumour cells or kidney tissue, or from an individual, or group of individuals, not suffering from kidney cancer; and
c) establishing the presence, or absence, of a kidney tumour based on the established up- or down regulation of said one or more genes.

17. Method according to claim 16, wherein said method is an ex vivo and/or in vitro method.

18. Method according to claim 16, wherein determining expression of said one or more genes comprises determining mRNA expression.

19. Method according to claim 16, wherein determining expression of said one or more genes comprises determining protein levels.

20. Method according to claim 16, wherein said one or more is selected from the group consisting of two or more; three or more; four or more and five.

21. Method according to claim 16, wherein establishing the presence, or absence, of a tumour further comprises establishing metastasis or no metastasis.

22. Method according to claim 16, wherein said kidney cancer is renal cell carcinoma.

23. Use of expression analysis of one or more genes chosen from the group consisting of NDUFA4l2, ANGPTL4, EGLN3, PTHLH, and ATP6V1B1 for establishing the presence, or absence, of a kidney tumour in a human individual suspected of suffering from kidney cancer.

24. Use according to claim 23, wherein said expression analysis is ex vivo and/or in vitro.

25. Use according to claim 23, wherein said one or more is selected from the group consisting of two or more, three or more, four or more, and five.

26. Use according to claim 23, wherein said kidney cancer is renal cell carcinoma

27. Kit of parts for establishing the presence, or absence, of a kidney tumour in a human individual suspected of suffering from kidney cancer:
expression analysis means for determining the expression of one or more genes one or more genes chosen from the group consisting of NDUFA4l2, ANGPTL4, EGLN3, PTHLH, and ATP6V1B1; and
instructions for use.

28. Kit of parts according to claim 27, wherein said expression analysis means comprises mRNA expression analysis means, preferably for PCR, rtPCR or NASBA.

29. Kit of parts according to claim 27, wherein said one or more is selected from the group consisting of two or more, three or more, four or more, and five.

30. Kit of parts according to claim 27, wherein said kidney cancer is renal cell carcinoma.
